(19) United States
(12) Patent Application Publication
(10) Pub. No.: US 2014/0224247 A1
(43) Pub. Date: Aug. 14, 2014

(54) RESPIRATORY THERAPY DEVICE FOR HOME USE

(71) Applicant: HILL-ROM SERVICES PTE. LTD., Singapore (SG)

(72) Inventor: Hee Choon TAN, Singapore (SG)

(73) Assignee: HILL-ROM SERVICES PTE. LTD., Singapore (SG)

(21) Appl. No.: 13/790,264

(22) Filed: Mar. 8, 2013

Related U.S. Application Data
(60) Provisional application No. 61/764,890, filed on Feb. 14, 2013.

(51) Int. Cl. A61M 16/16; A61M 16/04; A61M 16/08; A61M 16/00; A61M 16/10

(52) U.S. Cl. CPC A61M 16/16; A61M 16/0066; A61M 16/105; A61M 16/1075; A61M 16/0875; A61M 16/0488
USPC 128/203.14; 128/205.12; 128/204.17; 128/203.12

(57) ABSTRACT

A respiratory therapy device in accordance with this disclosure includes a blower box and a patient circuit. The blower box is configured to provide aerosol and pressurized gas for delivery to a patient airway. The patient circuit is configured to deliver the aerosol and pressurized gas from the blower box to the patient airway.

### CROSS-REFERENCE TO RELATED APPLICATIONS

The present application claims the benefit, under 35 U.S.C. §119(e), of U.S. Provisional Application No. 61/764,890, which was filed Feb. 14, 2013 and which is hereby incorporated by reference here.

### BACKGROUND

The present disclosure is related to respiratory therapy devices. More specifically, the present disclosure is related to a respiratory therapy device that delivers therapeutic gas to a patient's airway.

Some respiratory therapy devices deliver pressurized gas to a patient's airway. Many such devices are configured to be coupled to pressurized gas canisters or pressurized gas lines that provide pressurized gas, often oxygen, to the respiratory therapy device for distribution to the patient. Pressurized gas canisters or pressurized gas lines are often available in acute-care settings, like a hospital. However, pressurized gas canisters and pressurized gas lines are not always available or convenient outside of acute-care settings, such as in a patient's home.

### SUMMARY

The present application discloses one or more of the features recited in the appended claims and/or the following features which, alone or in any combination, may comprise patentable subject matter:

According to the present disclosure, a respiratory therapy device may include a housing and a compressor. The compressor may be configured to pressurize atmospheric gasses.

In some embodiments, the compressor may be coupled to the housing for vibration relative to the housing. The respiratory therapy device may also include an air preparation unit. The air preparation unit may be mechanically coupled to the compressor for vibration therewith and may be pneumatically coupled to the compressor to receive the pressurized atmospheric gasses produced by the compressor. The air preparation unit may be configured to separate fluid from the pressurized atmospheric gasses at least in part due to vibration induced by the compressor.

In some embodiment, the air preparation unit may include a heater element and a mist separator. The heater element may be configured to warm the pressurized atmospheric gasses produced by the compressor. The mist separator may be configured to separate fluid from the warmed and pressurized atmospheric gasses.

In some embodiments, the respiratory therapy device may include a nebulizer unit mechanically coupled to the compressor for vibration therewith. The nebulizer unit may be pneumatically coupled to the air preparation unit and may be configured to nebulize the fluid separated from the pressurized atmospheric gasses by the mist separator. In some embodiments, the nebulizer may be pneumatically coupled to a medical solution reservoir and may be configured to nebulize liquid medicines in the medical solution reservoir.

In some embodiments, the respiratory therapy device may include a pressure control manifold pneumatically coupled to the compressor. The pressure control manifold may be configured to regulate the pressurized atmospheric gasses before delivery to a patient. In some embodiments, the pressure control manifold may be configured to regulate the pressurized atmospheric gasses in a first mode at a continuous pressure level and in a second mode at alternating pressure levels.

In some embodiments, the respiratory therapy device may include a patient circuit. The patient circuit may be pneumatically coupled to the nebulizer unit and pneumatically coupled to the pressure control manifold. The patient circuit may also be configured to be coupled to a patient airway to deliver regulated pressurized atmospheric gasses and nebulized liquids to the patient airway.

According to another aspect of the present disclosure, a respiratory therapy device may include a housing, a compressor, a medical solution reservoir, and a nebulizer unit. The compressor may be configured to pressurize atmospheric gasses and may be coupled to the housing for vibration relative to the housing. The medical solution reservoir may be configured to hold liquid medicines. The nebulizer unit may be pneumatically coupled to the medical solution reservoir.

In some embodiments, the nebulizer unit may be mechanically coupled to the compressor for vibration therewith. The nebulizer unit may be configured to nebulize the liquid medicines in the medical solution reservoir at least in part due to vibration induced by the compressor. In some embodiments, the nebulizer unit may include a nebulizing element that is pneumatically coupled to the compressor and driven by the pressurized atmospheric gasses provided by the compressor to nebulize the liquid medicines in the medical solution reservoir.

In some embodiments, the respiratory therapy device may include an air preparation unit. The air preparation unit may be pneumatically coupled to the compressor and may be configured to separate fluid from the pressurized atmospheric gasses provided by the compressor. The air preparation unit may be mechanically coupled to the compressor for vibration therewith. In some embodiments, the nebulizer unit may be pneumatically coupled to the air preparation unit and may be configured to nebulize the fluid separated from the pressurized atmospheric gasses by the air preparation unit.

In some embodiments, the respiratory therapy device may include a pressure control manifold. The pressure control manifold may be pneumatically coupled to the compressor and may be configured to regulate the pressurized atmospheric gasses before delivery to a patient. In some embodiments, the pressure control manifold may be configured to regulate the pressurized atmospheric gasses in a first mode at a continuous pressure level and in a second mode at alternating pressure levels.

In some embodiments, the respiratory therapy device may include a patient circuit. The patient circuit may be pneumatically coupled to the nebulizer unit and pneumatically coupled to the pressure control manifold. The patient circuit may be configured to be coupled to a patient airway to deliver regulated pressurized atmospheric gasses and nebulized liquid medicines to the patient airway.

According to another aspect of the present disclosure, a respiratory therapy device may include a housing and a compressor. The compressor may be configured to pressurize atmospheric gasses and may be coupled to the housing for vibration relative to the housing.

In some embodiments, the respiratory therapy device may include an air preparation unit and a nebulizer unit. The air preparation unit may be mechanically coupled to the compressor for vibration therewith and pneumatically coupled to the compressor to receive the pressurized atmospheric gasses produced by the compressor. The nebulizer unit may be mechanically coupled to the compressor for vibration therewith and pneumatically coupled to the air preparation unit. The air preparation unit may be configured to separate fluid from the pressurized atmospheric gasses provided by the compressor. The nebulizer unit may be configured to nebulize the fluid from the air preparation unit.

In some embodiments, the respiratory therapy device may also include a medical solution reservoir. The nebulizer unit may be pneumatically coupled to the medical solution reservoir and may be configured to nebulize liquid medicines in the medical solution reservoir.

In some embodiments, the respiratory therapy device includes a pressure control manifold. The pressure control manifold may be pneumatically coupled to the air preparation unit. The pressure control manifold may be configured to regulate the pressurized atmospheric gasses exiting the air preparation unit in a first mode at a continuous pressure level and in a second mode at alternating pressure levels. It is contemplated that the respiratory therapy device may include a patient circuit. The patient circuit may be pneumatically coupled to the nebulizer unit and to the pressure control manifold and may be configured to be coupled to a patient airway.

Additional features, which alone or in combination with any other feature(s), including those listed above and those listed in the claims, may comprise patentable subject matter and will become apparent to those skilled in the art upon consideration of the following detailed description of illustrative embodiments exemplifying the best mode of carrying out the invention as presently perceived.

### BRIEF DESCRIPTION OF THE DRAWINGS

The detailed description particularly refers to the accompanying figures in which:

FIG. 1 is a perspective view of a respiratory therapy device including a blower box and a patient circuit configured to deliver medicated aerosol and/or pressurized gas to a patient airway to provide respiratory therapies to the patient;

FIG. 2 is a diagrammatic view of the respiratory therapy device of FIG. 1; and

FIG. 3 is a diagrammatic view of a pneumatic circuit created between the atmosphere and the patient airway by the respiratory therapy device of FIGS. 1 and 2.

### DETAILED DESCRIPTION OF THE DRAWINGS

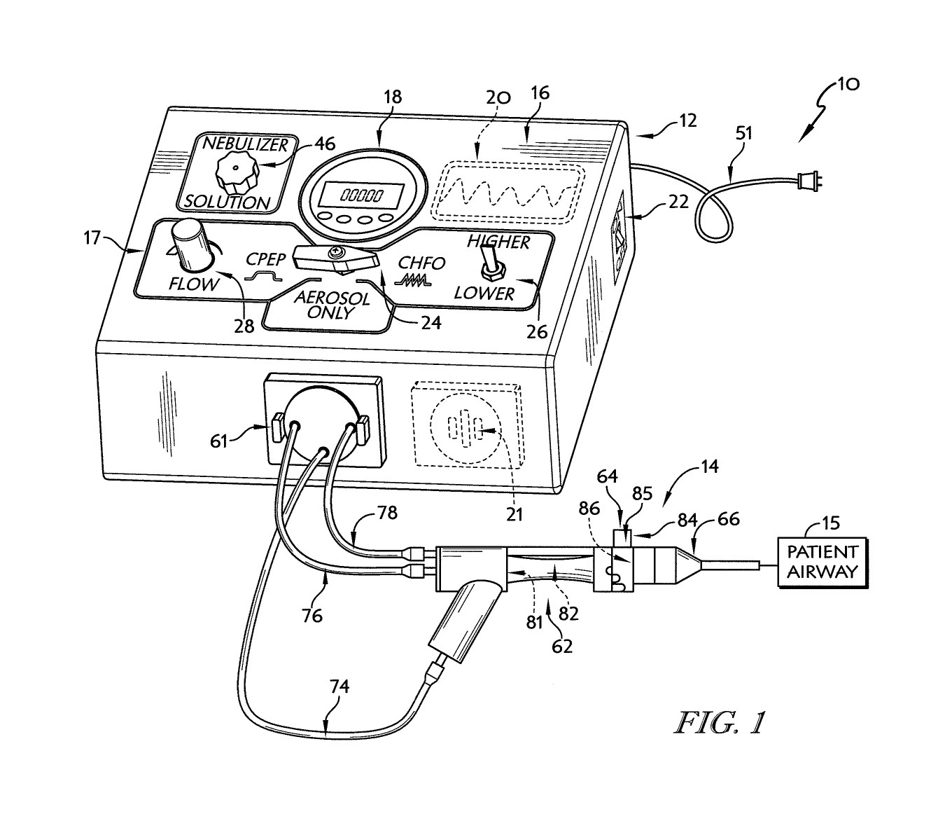

An illustrative respiratory therapy device 10 is configured for use in a non-acute setting, such as a patient's home, and includes a blower box 12 and a patient circuit 14 as shown in FIG. 1. The blower box 12 is configured to supply therapeutic gas for a patient's airway 15. The patient circuit 14 is coupled to the blower box 12 and extends to the patient's airway 15 to deliver the therapeutic gas to the patient's airway 15 from the blower box 12. The exemplary device 10 is configured to provide therapies for, among other things, enhancing normal mucus clearance and for addressing patchy atelectasis. Patchy atelectasis is a condition characterized by decreased aeration and collapse of multiple small areas of the lung.

The particular therapies provided by the illustrative device 10 include aerosol only therapy, CPEP therapy, and CHFO therapy as suggested in FIG. 1. When providing aerosol only therapy, the device 10 delivers medicated and/or humidified aerosol to the patient's airway 15 without applying pressure to the patient's airway 15. When providing CPEP (Continuous Positive Epiratory Pressure) therapy, the device 10 delivers the aerosol while continuously applying positive pressure to the patient's airway 15 to assist in holding open and expanding the patient's airways 15. When providing CHFO (Continuous High Frequency Oscillation) therapy, the device 10 delivers the aerosol while also applying oscillating pressure to the patient's airway 15 with continuous pulses of positive pressure.

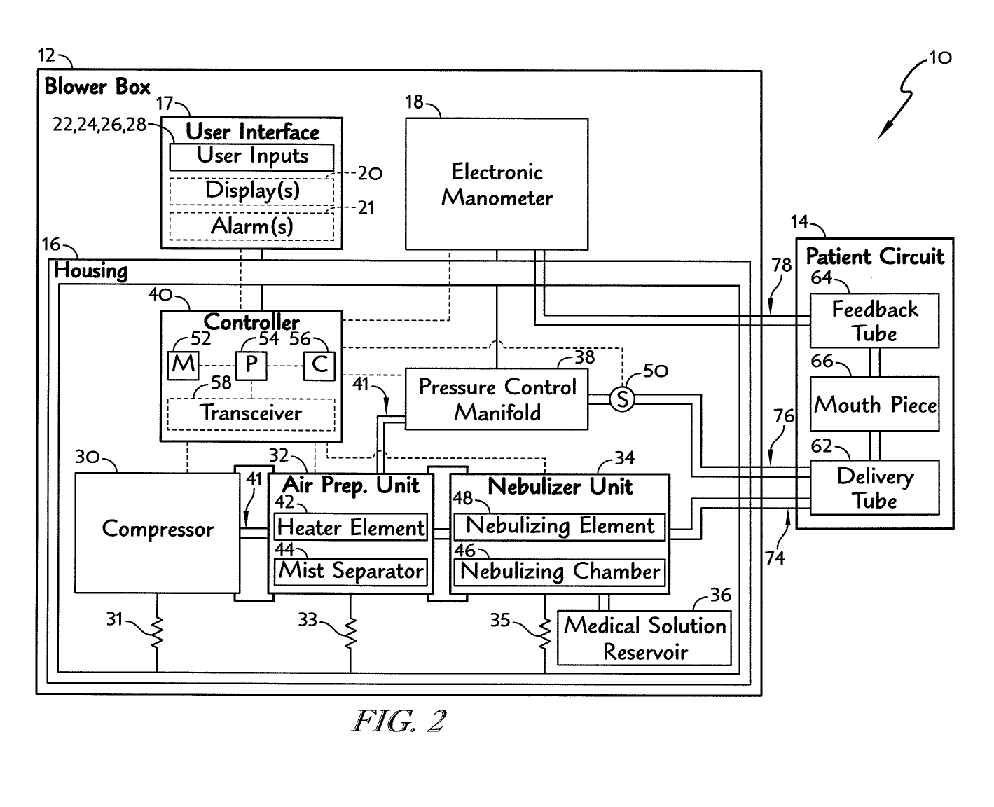

The blower box 12 is configured to produce the aerosol and any pressurize gas that is subsequently delivered to the patient by the patient circuit 14. The blower box 12 illustratively includes a housing 16, a user interface 17, and an electronic manometer 18 as shown in FIG. 1. The blower box 12 also includes a compressor 30 encased in the housing 16 and configured to drive some components 32, 34 of the blower box 12 by providing pressurized gas and vibration energy to the driven components 32, 34, as shown in FIG. 2. As a result of harnessing the vibration energy of the compressor 30 to drive components of the blower box 12 rather than just the pressurized gas, the size of the compressor 30 can be reduced. Reducing the size of the compressor 30 may allow for a relatively small blower box 12 adapted for use outside of acute-care settings.

The blower box 12 may also include an optional display 20 that is configured to show information about the operation of the device 10. The user interface 17 is coupled to the housing 16 and allows a user to adjust the operation of the blower box 12. The electronic manometer 18 is also coupled to the housing 16 and is configured to display the pressure exerted when a patient to exhales while a therapy is being applied.

The user interface 17 illustratively includes an on-off input 22, a therapy selector input 24, a high-low input 26, and a flow input 28 as shown in FIG. 1. The on-off input 22 is configured to power up or power down the blower box 12. The therapy selector input 24 is configured to select which therapy is to be provided by the device 10. The high-low input 26 is configured to increase and decrease the pressure and, in the case of CHFO, to increase and decrease the frequency of pressure oscillation provided by the device during a therapy. The flow input 28 is configured to increase and decrease flow provided by the device 10 during a therapy. In some embodiments, the user interface 17 may include an alarm speaker 21 encased in the housing 16 and configured to emit an alarm signal when a timed therapy session is complete, when therapy pressures are too small or too large, or when other event are detected.

Referring now to FIG. 2, the blower box 12 is shown diagrammatically to show the blower box components hidden inside the housing 16. In the illustrative embodiment, the blower box 12 includes the compressor 30, an air preparation unit 32, a nebulizer unit 34, a medical solution reservoir 36, a pressure control manifold 38, and a controller 40. Each of these pneumatic blower box components 30, 32, 34, 36, 38 are connected by a pneumatic line 41.

The compressor 30 is illustratively configured to pressurize atmospheric air from around the blower box 12 to provide pressurized therapeutic gas to a patient airway 15 as shown in FIG. 2. The use of the compressor 30 provides pressurized gas without requiring a pressurized gas canister or pressurized gas line. The compressor 30 is coupled to the housing 16 by an elastic connector 31 so that the compressor 30 vibrates relative to the housing 16 when the compressor 30 is running. In the illustrative embodiment, the elastic connector 31 is a rubber grommet (not shown) but, in other embodiments, may be a spring or another suitable connector. The compressor 30 is illustratively configured to be adjusted to provide more or less pressurized flow depending on the setting of the flow input 28.

The air preparation unit 32 is coupled to the compressor 30 along the pneumatic line 41 to receive the pressurized atmospheric air from the compressor 30 as shown in FIG. 2. The air preparation unit 32 is also mechanically coupled to the compressor 30 to vibrate with the compressor 30 when the compressor 30 is running. In addition to transferring vibration, the mechanical coupling of the air preparation unit 32 to the compressor 30 also provides a conductive thermal connection between the air preparation unit 32 and the compressor 30. Because both the air preparation unit 32 and the compressor 30 are located inside the housing 16, a convective thermal connection between the air preparation unit 32 and the compressor is also formed. Thus, wasted vibration energy and heat energy produced by the compressor 30 is passed to the air preparation unit 32.

The air preparation unit 32 illustratively includes a heater element 42 and a mist separator 44 as shown in FIG. 2. The heater element 42 is configured to warm up the pressurized atmospheric air from the compressor 30 to make the air more comfortable for inhalation by a patient. The pressurized atmospheric air from the compressor 30 is also heated using waste heat passed to the air preparation unit 32 via conduction and convection as discussed above. The use of waste heat to warm the pressurized atmospheric air reduces the amount of power required to be input into the heater element 42 to raise the air temperature to a predetermined level. The mist separator 44 is configured to separate/capture fluid (typically water) from the pressurized atmospheric gasses provided by the compressor 30. The mist separator 44 is driven, at least in part, by vibration induced in the air preparation unit 32 when the compressor 30 is running.

The heated and dried air produced by the air preparation unit 32 is sent on to the pressure control manifold 38 while removed fluids are sent on to the nebulizer unit 34 as suggested in FIG. 2. Because the air sent to the pressure control manifold 38 is dried, clogging and/or corrosion of valves and other components in the pressure control manifold may be avoided.

The air preparation unit 32 is also coupled to the housing 16 by an elastic connector 33. In the illustrative embodiment, the elastic connector 33 is a rubber grommet (not shown) but, in other embodiments, may be a spring or another suitable connector.

The nebulizer unit 34 is coupled to the air preparation unit 32 along the pneumatic line 41 as shown in FIG. 2. The nebulizer unit 34 receives both the fluid from the mist separator 44 and some of the pressurized atmospheric air provided to the air preparation unit 32 by the compressor 30. The nebulizer unit 34 is also pneumatically coupled to the medical solution reservoir 36 that is configured to hold liquid medicine. The nebulizer unit 34 is configured to nebulize (meaning to reduce to a fine spray) the fluid from the mist separator 44 and any liquid medicine contained in the medical solution reservoir 36. By nebulizing the fluid (typically water) from the mist separator 44, the nebulizer unit 34 provides humidity to the air passing through nebulizer unit 34 to make inhalation of the air more comfortable for a patient. By nebulizing the liquid medicine in the medical solution reservoir 36, the nebulizer unit 34 provides the medicine in a form that can be comfortably inhaled by a patient.

The nebulizer unit 34 is also mechanically coupled to the compressor 30 to vibrate with the compressor 30 when the compressor 30 is running as shown in FIG. 2. The nebulizer unit 34 illustratively includes nebulizing chamber 46 and a nebulizing element 48 as shown diagrammatically in FIG. 2. The nebulizing chamber 46 receives and holds liquid from the mist separator 44 and fluid medicine from the medical solution reservoir 36 to be neblized. The nebulizing element 48 reduces liquid in the chamber 46 to a fine mist and passes the mist on to an air stream directed to the patient circuit 14.

The nebulizing element 48 is illustratively driven mainly by the pressurized air provided by the compressor 30 through the air preparation unit 32 to create the medicated and/or humidified aerosol. However, the nebulizing element 48 is also driven, in part, by vibration of the nebulizer unit 34 induced by its connection to the compressor 30. By utilizing the vibration of the compressor 30 to drive the nebulizing element 48, less pressurized air is required to drive the nebulizing element 48 allowing for a smaller compressor 30 than would otherwise be needed to drive the nebulizing element 48 with pressurized air alone. In the illustrative embodiment, the nebulizing element 48 is made up of both a jet nebulizing component and a vibrating mesh nebulizing component. In other embodiments, the nebulizing element may include only a jet nebulizing component or only a vibrating mesh nebulizing component. In other embodiments, the nebulizing element may include other individual or combination nebulizing components.

The nebulizer unit 34 is illustratively coupled to the housing 16 by an elastic connector 35 as shown in FIG. 2. The elastic connector 35 allows the nebulizer unit 34 to vibrate with the compressor 30 when the compressor 30 is running. In the illustrative embodiment, the elastic connector 35 is a rubber grommet (not shown) but, in other embodiments, may be a spring or another suitable connector.

The pressure control manifold 38 is coupled to the air preparation unit 32 along the pneumatic line 41 as shown in FIG. 2. The pressure control manifold 38 includes a number of valves (not shown) and is configured to regulate the heated pressurized gas before delivery to a patient. In the illustrative embodiment, the pressure control manifold 38 is configured to regulate the heated pressurized gas in a first mode when the device 10 is delivering CPEP therapy and in a second mode when the device 10 is delivering CHFO therapy. In the first mode, the pressure control manifold 38 regulates pressure exiting the blower box 12 and sent to the patient circuit 14 to a continuous pressure level. In the second mode, the pressure control manifold 38 regulates pressure exiting the blower box 12 and sent to the patient circuit 14 at alternating pressure levels by pulsing the pressurized gas passed through the pressure control manifold 38 (typically between about 1 and 15 hertz).

The controller 40 is illustratively configured to adjust operation of the compressor 30, the air preparation unit 32, and the pressure control manifold 38 to provide the different therapies available from the device 10 based on the settings of the user interface 17 as suggested in FIG. 2. The controller 40 is coupled to the user interface 17, the manometer 18, the compressor 30, the air preparation unit 32, the nebulizer unit 34, the pressure control manifold 38, and a sensor unit 50 as shown in FIG. 2. Power from a power outlet (not shown) is distributed by the controller 40 to each powered component 17, 18, 30, 32, 32, 34, 38, and 50 from a power cord 51 that extends out of the housing 16 shown in FIG. 1. The sensor unit 50 is illustratively configured to detect pressure and temperature of the gas in the pneumatic line 41 exiting the pressure control manifold 38 being sent to the patient circuit 14. In some embodiments, the sensor unit 50 may also detect relative humidity and/or other characteristics of the gas in the pneumatic line 41 exiting the pressure control manifold 38.

In the illustrative embodiment, the controller 40 includes a memory 52, a processor 54, and a clock 56 as shown in FIG. 2. The memory 52 is illustratively coupled to the processor 54 and contains instructions that are performed by the processor 54. The processor 54 executes the instructions written on the memory 52 and sends data from the manometer 18 and the sensor unit 50 for storage and, in some embodiments, later use. The clock 56 is coupled to the processor 54 and provides time stamps to the processor 54 to be associated with stored information recorded in the memory 52 by the processor 54.

In operation, the controller 40 is configured to control the heater element 42 of the air preparation unit 32 and the pressure control manifold 38 during operation of the respiratory therapy device 10 to maintain a predetermined air temperature and air pressure provided to the patient circuit 14. More specifically, the controller 40 adjusts operation of the heater element 42 and the settings of the pressure control manifold 38 in response to temperature and pressure information received from the sensor unit 50 so that air provided to a patient is conditioned to a predetermined temperature and pressure.

The processor 54 is also configured to store the pressure of exhalation detected by the manometer 18 and the pressure of air exiting the pressure control manifold 38 provided by the sensor unit 50 over time to the memory 52. The data recorded in the memory 52 may be displayed on the optional display 20 coupled to the housing 16 to allow a caregiver to monitor or review therapies applied to a patient. In some embodiments, the controller 40 may also include an optional transceiver 58 coupled to the processor 54 and configured to communicate the recorded data to an external storage device, a computer, a network, or the like for storage and analysis.

The patient circuit 14 is illustratively coupled to the blower box 12 by a multiple conduit connector apparatus 61 as shown in FIG. 1. The multiple conduit connector apparatus 61 and a method of using the apparatus 61 is described in detail in U.S. Publication Number 2011/0100364 A1 to Faram. U.S. Publication Number 2011/0100364 A1 is hereby incorporated by reference herein in its entirety, except in parts where it irreconcilably conflicts with the drawings and description herein.

The patient circuit 14 also illustratively includes a delivery tube 62, a feedback tube 64, and a mouth piece 66 as shown in FIGS. 1 and 2. The delivery tube 62 is coupled to the nebulizer unit 34 via an aerosol hose 74 and the pressure control manifold 38 via a pressure hose 76 as shown in FIGS. 1 and 2. The feedback tube 64 is coupled to the manometer 18 and to the atmosphere 11 via a vent hose 78 as shown in FIGS. 1 and 2. The mouth piece 66 is coupled to both the delivery tube 62 and to the feedback tube 64. The mouth piece 66 is configured to be engaged by a patient's mouth to couple the patient circuit 14 to the patient's airway as suggested in FIG. 1.

The exemplary delivery tube 62 includes a venture tube 68 with pressure ports 71 and 72 as shown in FIG. 1. Pressure sensors 81, 82 are mounted in the pressure ports 71, 72 and are coupled to the controller 40 to allow flow through the delivery tube 62 to be calculated, displayed, and/or stored for caregiver monitoring or review of therapy.

The exemplary feedback tube 64 includes a resistance assembly 84 as shown in FIG. 1. The resistance assembly 84 includes an adjustment knob 85 that is rotatable to adjust a restriction 86 inside the resistance assembly 84. By rotating the knob 85, a caregiver or patient can increase or decrease the pressure required to be exerted by a patient exhaling during therapy sessions with the device 10.

In some embodiments, the patient circuit 14 may optionally be replaced with a patient circuit described in one of U.S. Pat. Nos. 8,051,854 B2, 7,909,033 B2, or 7,191,780 each to Faram. U.S. Pat. Nos. 8,051,854 B2, 7,909,033 B2, and 7,191,780 are each hereby incorporated by reference herein in their entirety, insofar as they disclose alternative patient circuits for use in a respiratory therapy device.

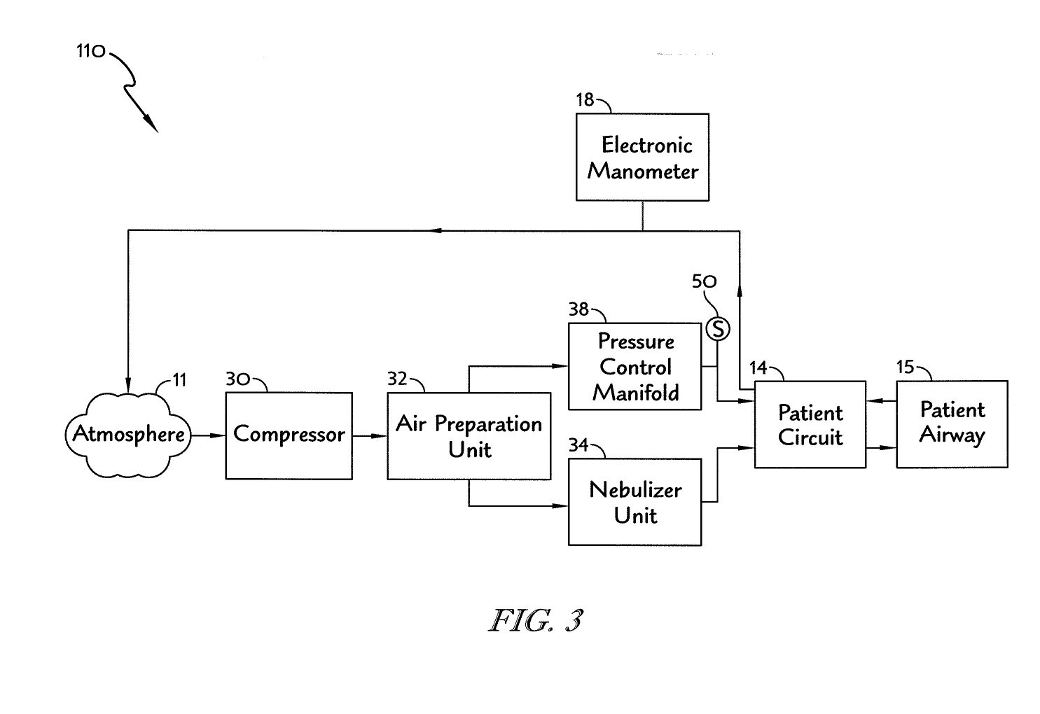

Referring now to FIG. 3, an illustrative pneumatic circuit 110 formed by the device 10 when providing therapy to a patient's airway 15 is shown. When CPEP or CHFO therapy is provided by the device 10, air from the atmosphere 11 is gathered by the compressor 30 and pressurized before being sent on to the air preparation unit 32. After heat is added to the pressurized air and fluid is separated from the heated pressurized air in the air preparation unit 32, a first portion of the heated pressurized air is passed on to the nebulizer unit 34. A second portion of the heated pressurized air is passed on to the pressure control manifold 38 to provide pressurized air for CPEP or CHFO therapy to the patient circuit 14 as shown in FIG. 3.

The first portion of heated pressurized air passed to the nebulizer unit 34 is used to nebulize fluid and liquid medicine provided to the nebulizer and to drive the resulting spray to the patient circuit 14. When the device 10 is providing the aerosol only therapy, the spray from the nebulizer unit 34 is the only therapy gas provided to a patient's airway and the second portion of the heated pressurized air is blocked from passing to the patient circuit 14.

After the patient has inhaled the spray from the nebulizer unit 34 and (sometimes) the pressurized gas from the pressure control manifold 38, the patient exhales back through the patient circuit 14 as suggested by the dashed line in FIG. 3. The exhaled gas is passed through the feedback tube 64 of the patient circuit 14 and out to the atmosphere 11.

Although certain illustrative embodiments have been described in detail above, variations and modifications exist within the scope and spirit of this disclosure as described and as defined in the following claims.

1. A respiratory therapy device comprising
a housing,
a compressor configured to pressurize atmospheric gasses and coupled to the housing for vibration relative to the housing, and
an air preparation unit mechanically coupled to the compressor for vibration therewith and pneumatically coupled to the compressor to receive the pressurized atmospheric gasses produced by the compressor, wherein the air preparation unit is configured to separate fluid from the pressurized atmospheric gasses at least in part due to vibration induced by the compressor.

2. The respiratory therapy device of claim 1, wherein the air preparation unit includes a heater element configured to warm the pressurized atmospheric gasses produced by the compressor and a mist separator configured to separate fluid from the warmed and pressurized atmospheric gasses.

3. The respiratory therapy device of claim 1, further comprising a nebulizer unit mechanically coupled to the compressor for vibration therewith.

4. The respiratory therapy device of claim 3, wherein the nebulizer unit is pneumatically coupled to the air preparation unit and is configured to nebulize the fluid separated from the pressurized atmospheric gasses by the mist separator.

5. The respiratory therapy device of claim 3, wherein the nebulizer is pneumatically coupled to a medical solution reservoir and is configured to nebulize liquid medicines in the medical solution reservoir.

6. The respiratory therapy device of claim 3, further comprising a pressure control manifold pneumatically coupled to the compressor and configured to regulate the pressurized atmospheric gasses before delivery to a patient.

7. The respiratory therapy device of claim 6, wherein the pressure control manifold is configured to regulate the pressurized atmospheric gasses in a first mode at a continuous pressure level and in a second mode at alternating pressure levels.

8. The respiratory therapy device of claim 6, further comprising a patient circuit pneumatically coupled to the nebulizer unit, pneumatically coupled to the pressure control manifold, and configured to be coupled to a patient airway to deliver regulated pressurized atmospheric gasses and nebulized liquids to the patient airway.

9. A respiratory therapy device comprising
a housing,
a compressor configured to pressurize atmospheric gasses and coupled to the housing for vibration relative to the housing,
a medical solution reservoir configured to hold liquid medicines, and
a nebulizer unit mechanically coupled to the compressor for vibration therewith and pneumatically coupled to the medical solution reservoir, wherein the nebulizer unit is configured to nebulize the liquid medicines in the medical solution reservoir at least in part due to vibration induced by the compressor.

10. The respiratory therapy device of claim 9, wherein the nebulizer unit includes a nebulizing element pneumatically coupled to the compressor and driven by the pressurized atmospheric gasses provided by the compressor to nebulize the liquid medicines in the medical solution reservoir.

11. The respiratory therapy device of claim 9, further comprising an air preparation unit pneumatically coupled to the compressor and configured to separate fluid from the pressurized atmospheric gasses provided by the compressor.

12. The respiratory therapy device of claim 11, wherein the air preparation unit is mechanically coupled to the compressor for vibration therewith.

13. The respiratory therapy device of claim 11, wherein the nebulizer unit is pneumatically coupled to the air preparation unit and is configured to nebulize the fluid separated from the pressurized atmospheric gasses by the air preparation unit.

14. The respiratory therapy device of claim 9, further comprising a pressure control manifold pneumatically coupled to the compressor and configured to regulate the pressurized atmospheric gasses before delivery to a patient.

15. The respiratory therapy device of claim 14, wherein the pressure control manifold is configured to regulate the pressurized atmospheric gasses in a first mode at a continuous pressure level and in a second mode at alternating pressure levels.

16. The respiratory therapy device of claim 14, further comprising a patient circuit pneumatically coupled to the nebulizer unit, pneumatically coupled to the pressure control manifold, and configured to be coupled to a patient airway to deliver regulated pressurized atmospheric gasses and nebulized liquid medicines to the patient airway.

17. A respiratory therapy device comprising
a housing,
a compressor configured to pressurize atmospheric gasses and coupled to the housing for vibration relative to the housing,
an air preparation unit mechanically coupled to the compressor for vibration therewith and pneumatically coupled to the compressor to receive the pressurized atmospheric gasses produced by the compressor, and
a nebulizer unit mechanically coupled to the compressor for vibration therewith and pneumatically coupled to the air preparation unit,
wherein the air preparation unit is configured to separate fluid from the pressurized atmospheric gasses provided by the compressor and the nebulizer unit is configured to nebulize the fluid from the air preparation unit.

18. The respiratory therapy device of claim 17, further comprising a medical solution reservoir, the nebulizer unit pneumatically coupled to the medical solution reservoir and configured to nebulize liquid medicines stored in the medical solution reservoir.

19. The respiratory therapy device of claim 17, further comprising a pressure control manifold pneumatically coupled to the air preparation unit and configured to regulate the pressurized atmospheric gasses exiting the air preparation unit in a first mode at a continuous pressure level and in a second mode at alternating pressure levels.

20. The respiratory therapy device of claim 19, further comprising a patient circuit pneumatically coupled to the nebulizer unit and to the pressure control manifold, the patient circuit configured to be coupled to a patient airway.
